(19) United States
(12) Patent Application Publication
(10) Pub. No.: US 2009/0010892 A1
(43) Pub. Date: Jan. 8, 2009

(54) Hyperlipidemia Treatment Agent

(75) Inventor: Takashi Masuda, Tokyo (JP)

Correspondence Address: EDELL, SHAPIRO & FINNAN, LLC, 1901 RESEARCH BLVD., SUITE 400, ROCKVILLE, MD, 20850-3164, US

(73) Assignee: Toa Pharmaceutical Co., Ltd., Tokyo (JP)

(21) Appl. No.: 11/774,172

(22) Filed: Jul. 6, 2007

(51) Int. Cl. A61K 35/74; A61P 3/06

(52) U.S. Cl. 424/93.4

(57) ABSTRACT

To provide a cholesterolemia treatment agent comprising, as an active ingredient, a bacterial mixture of three bacterial types consisting of lactic acid bacteria, butyric acid bacteria, and glycation bacteria which are effective in lowering serum cholesterol. And to provide a triglyceridemia treatment agent comprising, as an active ingredient, a bacterial mixture of three bacterial types consisting of lactic acid bacteria, butyric acid bacteria, and glycation bacteria which are effective in lowering triglycerides.

### BACKGROUND OF THE INVENTION

1. Field of the Invention

Hyperlipidemia is a condition in which the serum cholesterol, triglycerides and the like are raised. In addition to hyperlipidemia, high blood pressure, diabetes and the like are called lifestyle diseases. These diseases are a result of a lifestyle of high calorie, high fat foods, and lack of exercise.

Hyperlipidemia, high blood pressure, and diabetes are diseases which often appear together, and in addition, when there are the added risks of smoking and obesity, atherosclerosis is dramatically accelerated.

In coronary atherosclerosis of the heart, atheroma plaques which contain a large amount of cholesterol readily form in the coronary arteries, and the instability and rupture of these plaques can result in the occurrence of acute myocardial infarctions. In addition, with familial hyperlipidemia which is a result of genetic factors, there is a hetero-type in which the gene is inherited from one parent and a homo-type in which the gene is inherited from both parents. Familial hyperlipidemia of the hetero-type is present at a rate of approximately 1 in 500 people, and the homo-type is present in approximately 1 in 1,000,000 people.

The characteristic of this disease is that the serum cholesterol value is high at greater than 260 mg/dl. The Achilles tendon can thicken, and there can be cholesterol deposits onto the skin and eyelids called xanthomas.

2. Description of the Related Art

In general, treatment of hyperlipidemia is with a diet and exercise regimen. Biologically active substances contained in food products include those that affect cholesterol metabolism and lipid metabolism, and these can lower cholesterol and triglyceride values. Vitamin E, dietary fiber, and polyunsaturated fatty acids are good examples of such substances. However, lactic acid bacteria or lactic acid bacteria yogurt are also known to have an anti-hyperlipidemia effect. The mechanism for cholesterol suppression has been studied looking at different aspects, but the most frequently reported is the inhibition of absorption of cholesterol. In particular, there are theories such as absorption of cholesterol by the administered probiotics bacteria, suppression of absorption by conversion to coprostanol, and reduced micelle formation of the cholesterol due to bile acid absorption or un-suturing by bile acid.

The reported types of bacteria having cholesterol lowering action are bifidus bacteria, lactic acid cocci, lactobacillus, and the like or a yogurt created from these bacteria. Although there are many reports, there are some reports that find no effect. One factor may be that many of the reports use one type of bacteria, and the action may be weak, resulting in poor reproducibility.

### SUMMARY OF THE INVENTION

The present invention relates to a hypercholesterolemia treatment agent comprising, as an active ingredient, a live bacteria mixture or a dead bacteria mixture of 3 types of bacteria consisting of lactic acid bacteria, butyric acid bacteria, and glycation bacteria.

The problem to be solved by the present invention is to provide a probiotics formulation which is effective in treatment of cholesterolemia.

Based on relevant knowledge, the present inventors found that with having just one probiotics bacteria there was low reproducibility of anti-cholesterol action, but with a combination of lactic acid bacteria, butyric acid bacteria, and glycation bacteria, good cholesterol lowering action was confirmed.

In other words, the present invention relates to

1) A cholesterolemia treatment agent comprising, as an active ingredient, a bacterial mixture of three bacterial types consisting of lactic acid bacteria, butyric acid bacteria, and glycation bacteria which are effective in lowering serum cholesterol.
2) A triglyceridemia treatment agent comprising, as an active ingredient, a bacterial mixture of three bacterial types consisting of lactic acid bacteria, butyric acid bacteria, and glycation bacteria which are effective in lowering triglycerides.

By oral administration of a probiotics formulation of the present invention, treating of cholesterolemia is anticipated.

### BRIEF DESCRIPTION OF THE DRAWINGS

FIG. 1 shows a Change in triglycerides in the blood of elderly people before and after administration of BIO3.

### DESCRIPTION OF THE PREFERRED EMBODIMENTS

Below, the present invention is described in detail.

The cholesterol treatment substance of the present invention is characterized as a probiotics formulation.

The probiotics formulation of the present invention is Bio three (registered trademark) or a Bio three tablet (registered trademark).

For the probiotics formulation of the present invention, a single formulation of butyric acid bacteria, lactic acid bacteria, or glycation bacteria and a mixture formulation of butyric acid bacteria, lactic acid bacteria, and glycation bacteria are produced. Butyric acid bacteria, lactic acid bacteria, and glycation bacteria are each cultured, and after completing culturing, the bacteria are collected by centrifugation, and mixed with a stabilizer, and this is freeze dried. After drying, this is pulverized and mixed with a preferred base such as cornstarch, potato starch, dextrin, and the like. Butyric acid bacteria formulation, lactic acid formulation, glycation bacteria formulation, and a mixture formulation of butyric acid bacteria, lactic acid bacteria, and glycation bacteria are produced.

The anti-hyperlipidemia agent of the present invention is a probiotics formulation of lactic acid bacteria, butyric acid bacteria, and glycation bacteria. In other words, butyric acid bacteria, lactic acid bacteria, and glycation bacteria are mixed, and as a powder, fine granule, or granule, they can be used within a concentration range of 10 mg to 1000 mg.

The extension of dialysis introduction of the present invention is anticipated as a treatment for anti-hyperlipidemia by lowering hypercholesterolemia, hypertriglyceridemia, and the like.

### Embodiment 1

The hypercholesterolemia animal model was created as follows. In other words, a hypercholesterolemia model rat was produced by providing high cholesterol foods of cholesterol, cholic acid, milk casein, sucrose, coconut hydrogenated oil, KC flock, and Avicel cellulose to SD strain male rats. A butyric acid bacteria formulation, lactic acid bacteria formulation, glycation bacteria formulation or a mixture formulation of butyric acid bacteria, lactic acid bacteria, and glycation bacteria was mixed into the high cholesterol foods described above and given as feed. A control group was given a feed mixed with starch. Unlimited feed was provided for 14 days. During the experiment, weight, amount of feed intake, and general conditions were observed. One day prior to experiment completion, the rats were made to a fast. On the last day, the rats were killed and blood was taken from the abdominal aorta. Total cholesterol, HDL cholesterol, LDL cholesterol, triglycerides, phospholipids, total lipid, and atherosclerosis index were measured. The averages and standard deviations for resulting the measurements were calculated. This was compared with the control group using a student's t-test.

The triglyceridemia animal model was created as follows. Hypertriglyceridemia rats were made by giving high fat foods of casein, fructose, coconut hydrogenated oil, cellulose, and white fish to SD strain male rats. A butyric acid bacteria formulation, lactic acid bacteria formulation, glycation bacteria formulation, or a mixture formulation of butyric acid bacteria, lactic acid bacteria, and glycation bacteria was mixed with the high triglyceride foods and given as feed. The method of raising the rats and observation were the same as described previously.

The effect on the cholesterolemia model was as follows. There was no significant difference in the general condition, weight change, and amount of feed intake as compared with the control group. In addition, in all groups, there were no deaths. With regard to the blood biochemicals, with the butyric acid bacteria formulation, lactic acid bacteria formulation, and glycation bacteria formulation, there was no significant difference in all of the test items as compared to the control group. On the other hand, with the mixture formulation of butyric acid bacteria, lactic acid bacteria, and glycation bacteria, the total cholesterol, LDL cholesterol, phospholipids, total lipids, and atherosclerosis index were significantly reduced (p<0.05) as compared to the control group.

The effect on the triglyceridemia model was as follows. There were no significant differences with the control group in the general condition, weight change, and amount of feed intake. In all groups, there were no deaths. For the blood biochemicals, the butyric acid bacteria formulation, lactic acid bacteria formulation, and glycation bacteria formulation saw no significant difference in all of the test items with the control group. On the other hand, with the mixture formulation of butyric acid bacteria, lactic acid bacteria, and glycation bacteria, triglycerides were significantly lower than the control group (p<0.05).

TABLE 1
Effect of test sample on cholesterolemia rat

| Test | Total cholesterol (mg/dl) | LDL cholesterol (mg/dl) |
|---|---|---|
| C. butyricum TO-A | 224.6 ± 40.1 | 224.6 ± 40.1 |
| S. faecalis T-110 | 216.7 ± 32.5 | 216.7 ± 32.5 |
| B. mesentericus | 204.1 ± 30.2 | 204.1 ± 30.2 |
| TO-A | | |
| Mixture* | 175.1 ± 23.2 | 175.1 ± 23.2 |
| Control | 254.4 ± 58.3 | 254.4 ± 58.3 |

*Mixed of S. faecalis T-110, C. butyricum TO-A, B. mesentericus TO-A

TABLE 2
Effect of test sample on triglyceridemia rat

| Test | Triglyceride (mg/dl) |
|---|---|
| C. butyricum TO-A | 197.7 ± 26.8 |
| S. faecalis T-110 | 179.6 ± 45.6 |
| B. mesentericus TO-A | 191.2 ± 49.9 |
| Mixture* | 115.1 ± 12.8 |
| Control | 190.3 ± 34.3 |

### Embodiment 2

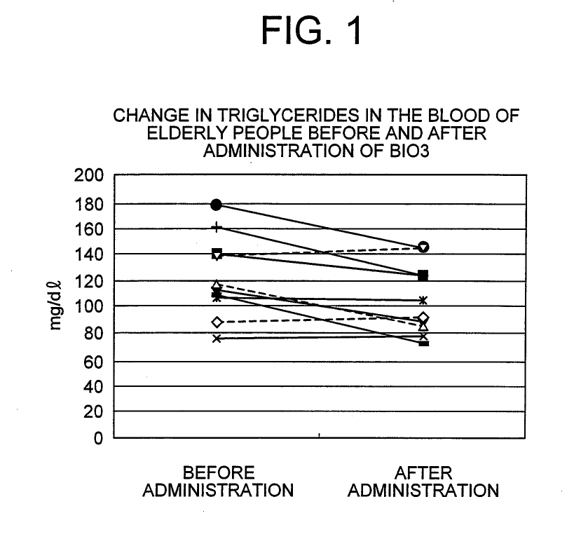

Probiotics BIO3 was administered over 2 weeks 3 times a day at 1 g each time to 10 elderly people. The patients had an average age of 82.5 and had chronic constipation, but had good food intake. As a result, there was a significant decline in triglycerides compared before and after administration of BIO3. (FIG. 1)

What is claimed is:

1. A cholesterolemia treatment agent comprising, as an active ingredient, a bacterial mixture of three bacterial types consisting of lactic acid bacteria, butyric acid bacteria, and glycation bacteria which are effective in lowering serum cholesterol.

2. A triglyceridemia treatment agent comprising, as an active ingredient, a bacterial mixture of three bacterial types consisting of lactic acid bacteria, butyric acid bacteria, and glycation bacteria which are effective in lowering triglycerides.
